(19) United States
(12) Patent Application Publication
(10) Pub. No.: US 2010/0318044 A1
(43) Pub. Date: Dec. 16, 2010

(54) PORTABLE URINE BAG

(75) Inventor: Yoshiharu TAKAYAMA, Tokyo (JP)

Correspondence Address: HOLTZ, HOLTZ, GOODMAN & CHICK PC, 220 Fifth Avenue, 16TH Floor, NEW YORK, NY, 10001-7708, US

(73) Assignee: KABUSIKI-KAISHA MUSH, Tokyo (JP)

(21) Appl. No.: 12/685,740

(22) Filed: Jan. 12, 2010

(30) Foreign Application Priority Data
Jun. 10, 2009 (JP) 2009-139392

(51) Int. Cl. A61F 5/44

(52) U.S. Cl. 604/317

(57) ABSTRACT

A urine bag, which is folded flat so that it is compact and ready for use, has a water impermeable bag portion and a flexible mouse portion secured at an upper opening of the bag portion. The flexible mouse portion surrounds at least a portion of the upper opening of the mouse portion and has shape preserving capability. The mouse portion includes left and right wings which can be displaced around a first central folding line to open or close the upper opening, and left and right tongues. The tongues can be displaced together with the wings around a second central folding line which is an extension of the first central folding line. The mouse portion also has a pair of symmetrical folding lines extending diagonally from the intersection of the contour of tongues and the upper edges of the wings to the first central folding line.

### FIELD OF THE INVENTION

The present invention relates generally to a urine bag to be used in a compartment of vehicles in traffic congestion, more particularly to a portable urine bag convenient for reliably receiving and storing the urine to be delivered therein, and which can be discarded easily.

### BACKGROUND OF THE INVENTION

When making leisure with children by car, it is often difficult to take a rest to go to the toilet because of the traffic congestion. In this connection, it is also necessary to carry urine bags provided against an emergency.

The urine bag known in the art may have a cylindrical mouse portion for keeping a degree of the shape preserving capability of the upper opening of the bag portion of the bag (see the publication 1) in order to keep the opening of the bag opened for receiving urine reliably in dependence on the condition of the vehicle.

The urine bag can be used not only in the compartment of vehicles, but also in outing so that it is necessary to provide the compact and portable urine bag. These compact and portable characteristics of the bag are also desirable in the standpoint of the physical distribution cost of the bags. In this connection, a bag including a foldable mouse portion to be attached to the opening of the bag portion of the urine bag had been developed (see the publication 2).

However, the bag having a sufficient shape preserving capability of the mouse portion upon used as well as some portability on the unused condition had not been realized.

### PUBLICATIONS ON THE ART

### Patents or Patent Applications

### 1. Japanese Utility Model Laid-Open Public Disclosure sho. 62-3144 (see pp. 1-2, and FIGS. 1 and 2)

### 2. Japanese Patent No. 3544367 (see pp. 1-10, and FIGS. 1-15)

### DISCLOSURE OF THE INVENTION

### Problem or Problems to be Solved by the Invention

It is the object of the present invention is to provide a urine bag which can in its unused condition be folded flat so that the bag is compact and convenient to be used any time you want. When it is intended to use the bag, the mouse portion thereof can be held stable in its cylindrical configuration. Further the structure of the bag is very simple so that the cost for making it can be reduced substantially.

### The Means for Solving the Problem or Problems

In order to solve the problem or problems as mentioned above, the urine bag of the present invention comprises a bag portion formed of any material inhibiting the permeation of water therethrough, and a mouse portion to be secured at an upper opening of the bag portion, wherein the mouse portion is formed of a sheet material surrounding at least a portion of the upper opening and having any suitable flexibility and shape preserving capability, wherein the mouse portion includes a pair of left and right wings, the wings can be displaced around the central folding line to open or close the upper opening, wherein the mouse portion further includes a pair of left and right tongues, the tongues can be displaced together with the wings around the second central folding line which is the extension of the central folding line, and wherein the mouse portion is also provided with a pair of symmetrical folding lines extending diagonally from the intersection of the contour of tongues and the upper edges of the wings to the central line.

Further, in the urine bag of the present invention, the central folding line, the second folding line, and the folding lines are all formed as mountain lines.

Moreover, in the urine bag of the present invention, each wing is provided with auxiliary folding line or lines extending parallel to the central folding line. The auxiliary folding line or lines are also formed as mountain lines. The folding line or lines are provided at its or their upper end or ends with a notch or notches.

In the urine bag of the present invention, the opposite inner faces of the upper opening of the bag portion are provided along their full width with a linear sealing fastener including a male part and a female part engageable and disengageable with each other. The ratio defined between the dimension between fully separated wings and the distance between the lower edge of the mouse portion and the fastener is to be determined as about 2:1.

Within the urine bag, a water absorbing means for absorbing urine is contained.

The mouse portion is formed of cardboard material.

The urine bag of the present invention comprises a bag portion formed of any material inhibiting the permeation of water therethrough, and a mouse portion to be secured at an upper opening of the bag portion, wherein the mouse portion is formed of a sheet material surrounding at least a portion of the upper opening and having any suitable flexibility and shape preserving capability, wherein the mouse portion includes a pair of left and right wings, the wings can be displaced around the central folding line to open or close the upper opening, wherein the opposite inner faces of the upper opening of the bag portion are provided along their full width with a linear sealing fastener including a male part and a female part engageable and disengageable with each other, and wherein the ratio defined between the dimension between fully separated wings and the distance between the lower edge of the mouse portion and the fastener is to be determined as about 2:1.

Further, in the urine bag of the present invention, the mouse portion further includes a pair of left and right tongues, the tongues can be displaced together with the wings around the second central folding line which is the extension of the central folding line, wherein the mouse portion is also provided with a pair of symmetrical folding lines extending diagonally from the intersection of the contour of tongues and the upper edge of the wing to the central line, and the central folding line, the second folding line, and the folding lines are all formed as mountain lines.

Moreover, in the urine bag of the present invention, each wing is provided with auxiliary folding line or lines extending parallel to the central folding line. The auxiliary folding line or lines are also formed as mountain lines. The folding line or lines are provided at its or their upper end or ends with a notch or notches.

Within the urine bag, a water absorbing means for absorbing urine is contained.

The mouse portion is formed of cardboard material.

### EFFECT TO BE OBTAINED FROM THE INVENTION

The urine bag of the present invention can in its unused condition be folded flat together with the wings to make the bag compact one, and the wings can be opened smoothly to their cylindrical configuration through folded along the central folding line, the second central folding line, and the auxiliary folding lines.

The upper opening of the bag can be maintained stable in its opened condition by folding the tongues provided on the upper edges of the wings inwardly or outwardly of the opening to remain the wings separated with each other.

Further the pieces provided between a pair of folding lines and the upper edges of the wings can also be folded inwardly of the bag. In this connection, when the pieces and tongues are folded inwardly of the bag, the tongues may also be served as a splash guard for preventing the urine from splashing out upon used. When the pieces are folded inwardly of the bag, and tongues are folded outwardly of the bag, only the tip portions of the tongues extend inwardly of the opening, and the other portions of the tongues extend outwardly from the opening. These outwardly extending portions can be used for grasping and supporting the bag when the bag is intended to be used for infants. In other words, a part of the mouse portion of the bag can be transformed by manipulating the tongues and the pieces conveniently. Further the part can be used for grasping the urine bag. Thus the urine bag of the high degree of freedom can be obtained.

After used the urine bag, the mouse portion of the bag can be folded again by only returning the tongues and the pieces in their unfolded condition.

When defined the dimensional ratio of the fastener defined between the dimension between wings fully displaced in their maximum and the distance between the lower edge of the mouse portion and the fastener is determined as about 2:1, and the linear sealing fastener 3 is not closed completely and held partially opened by remaining the unengaged portion, the bag can be opened easily by the one-touch operation through urging both ends of the fastener toward the unengaged portion. Thus, upon required to use the urine bag of the invention emergently, it can be used immediately without no confusion.

### BRIEF DESCRIPTION OF THE DRAWINGS

Embodiments of the present invention will now be described more fully with reference to the accompanying drawings in which:

FIG. 1 is an elevational view illustrating an embodiment of the urine bag of the present invention;

FIG. 2 is a perspective view illustrating the mouse portion widened to receive urine;

FIG. 3 is a perspective view illustrating the mouse portion in which the tongues are folded inwardly of the opening;

FIG. 4 is a perspective view illustrating the mouse portion in which the tongues are turned completely over the edge thereof;

FIG. 5(a) is a perspective view illustrating the mouse portion in which the tongues are urged in the vertical direction to rest on the pieces, and 5(b) is a cross-sectional view of the same;

FIG. 6 is a cross-sectional view along the line A-A of FIG. 1;

FIG. 7 is an enlarged cross-sectional view of the bag upon the fastener is closed;

FIG. 8 is a development of the mouse portion;

FIG. 9 is a perspective view upon the mouse portion is opened;

FIG. 10 is an elevational view of an embodiment of the urine bag containing water absorbing agent; and

FIG. 11 is a plan view illustrating the urine bag.

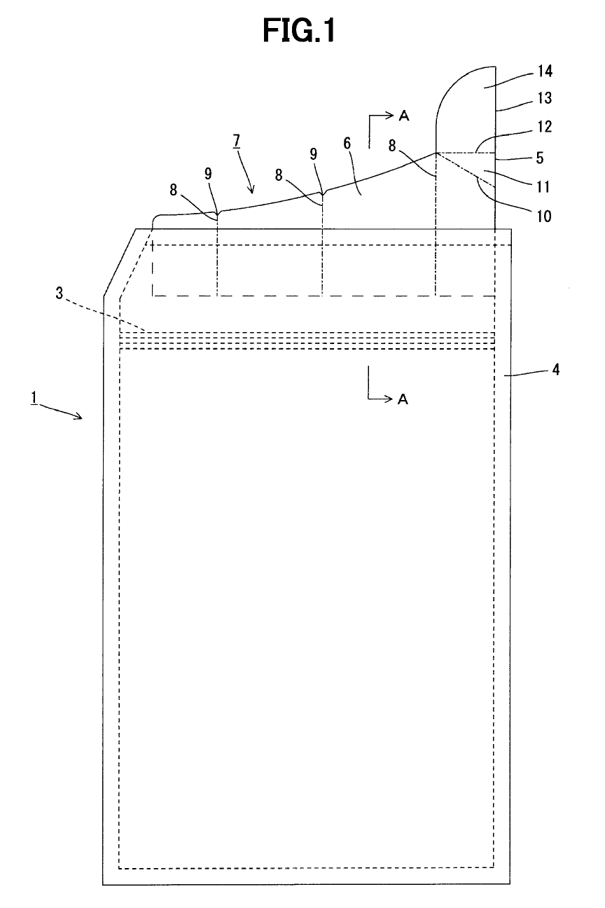

### DETAILED DESCRIPTION OF THE INVENTION

The best mode for providing the urine bag will now be described with reference to the attached drawings illustrating the concrete example thereof.

The urine bag 1 of the invention comprises a bag portion 1 and a mouse portion 7. The bag portion includes a pair of rectangular sheet materials for example of synthetic resin inhibiting the permeation of water therethrough. The sheet materials are closely placed one over the other, and heatsealed along the three sides thereof to form three heatsealed portions 4. The remaining side of the bag portion 1 is an upper opening 2. The opposite inner faces of the thus obtained bag portion are provided with a linear sealing fastener 3 including engageable and disengageable male and female parts 3a and 3b for sealing the opening 2 of the bag portion 1.

The mouse portion 7 which may preferably be formed of cardboard material is adapted to be secured on the inner faces of the upper opening 2 above the fastener 3. The mouse portion 7 includes a pair of left and right wings 6, 6 symmetrical with respect to the central folding line 5, and a pair of left and right tongues 14, 14 symmetrical with respect to the second central folding line 13 which is the extension of the central folding line 5. The upper opening 2 of the bag portion can be deformed together with wings 6, 6 and tongues 14, 14 to open or close the urine bag 1.

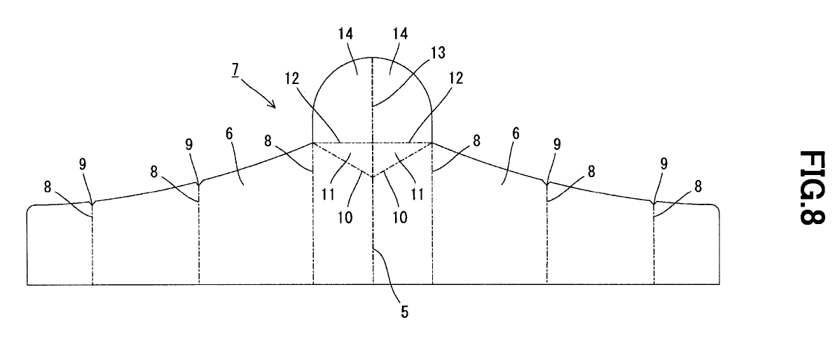

As can be seen from the development as shown in FIG. 8, each wing 6 of the mouse portion 7 is provided with three auxiliary folding lines 8 extending parallel to the central folding line 5. The line 8 of the wing 6 innermost to the central line 5 is the extension of the contour of the tongue. Each of the remaining two folding lines 8 further includes a notch 9 at the intersection with the upper edge of the mouse portion 7.

The mouse portion 7 is also provided with a pair of folding lines 10. Each folding line 10 extends diagonally from the intersection of the contour of the tongues 14 and the upper edge of the wing 6 to the central line 5.

The mouse portion 7 is also provided with a pair of second folding lines 12 extending between the intersections of the contour of the tongues 14 and the upper edge of the wings 6. Thus the tongues 14 can be folded into or out of the upper opening 2 of the bag 1 around the second folding lines 12 with respect to the wings 6.

The central folding line 5, the second central folding line 13, the auxiliary folding lines 8, and the folding lines 10 are all formed as mountain lines i.e. the lines to be folded to make crests direct toward the outer surface.

a) These mountain lines can be made on the blank sheet of the mouse portion 7 from the one surface thereof by any means known in the art. The mountain lines can be made by cutting-in, embossing, stamping, or making perforation therethrough.
b) These means for making mountain lines can be available depending on the material employed for the blank of the mouse portion 7.

The folding lines are shown in the attached drawings as alternate long and short dash lines.

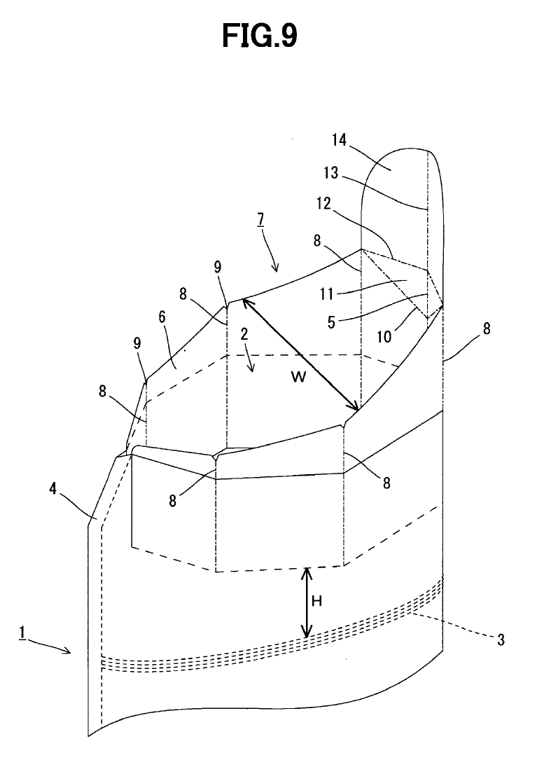

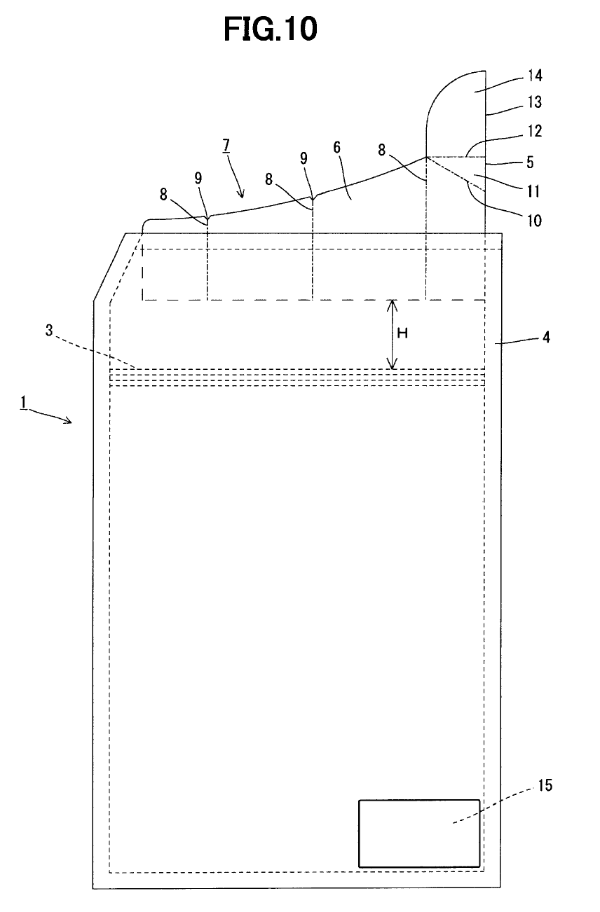

While the wings fully displaced in their maximum as shown in FIG. 9, the dimension between them are measured as (W). When the distance between the lower edge of the mouse portion 7 and the fastener 3 is defined as (H), the dimensional ratio of the fastener 3 defined as (W:H) is to be determined as about 2:1 or less.

The urine bag of the present invention of the structure as defined above will then be described with respect to the operation or function thereof.

The bag is, in its unused condition, folded flat and the mouse portion 7 thereof is also folded flat symmetrically around the central folding line 5 and the second central folding line 13. In this condition, the bag is compact and convenient for used any time you want.

When it is intended to use the bag, it was adapted to be opened by the disengagement of the sealing fastener 3 and widened the upper opening 2. Then the tongues 14, 14 and the wings 6, 6 are also spaced with each other to define the generally cylindrical envelope.

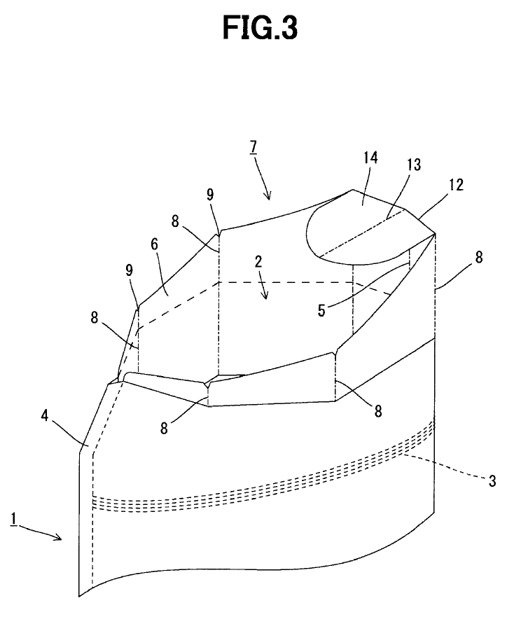

The distal or upper ends of tongues 14, 14 are then folded in the desired direction i.e. into or out of the upper opening 2 of the bag 1 around the second folding lines 12 as shown in FIG. 3. In this condition, the wings 6, 6 are spaced adequately by the presence of the tongues 14, 14. The tongues may also be served as a splash guard for preventing the urine from splashing out upon used.

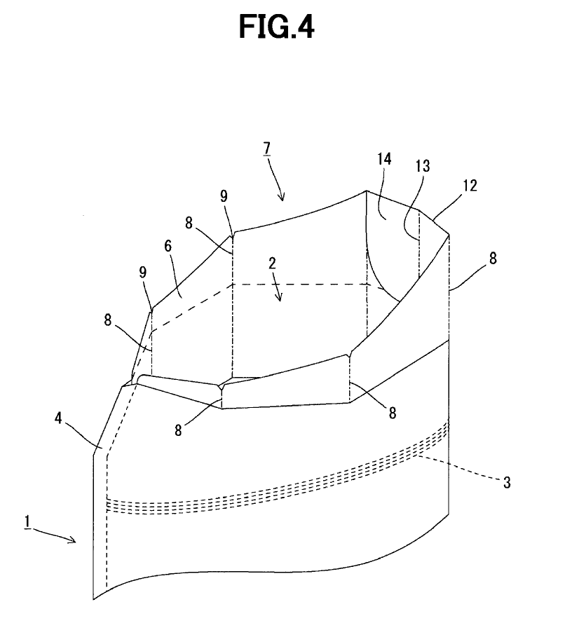

The tongues 14, 14 are then urged further downwardly to make contact with the surfaces of the wings 6, 6 as shown in FIG. 4. In this configuration, the spacing between the wings can be maintained, and the opening 2 of the bag 1 can be enlarged.

In the embodiment as shown in FIG. 4, although the tongues 14, 14 are folded inwardly of the opening 2, the tongues 14, 14 can also be folded outwardly of the opening 2.

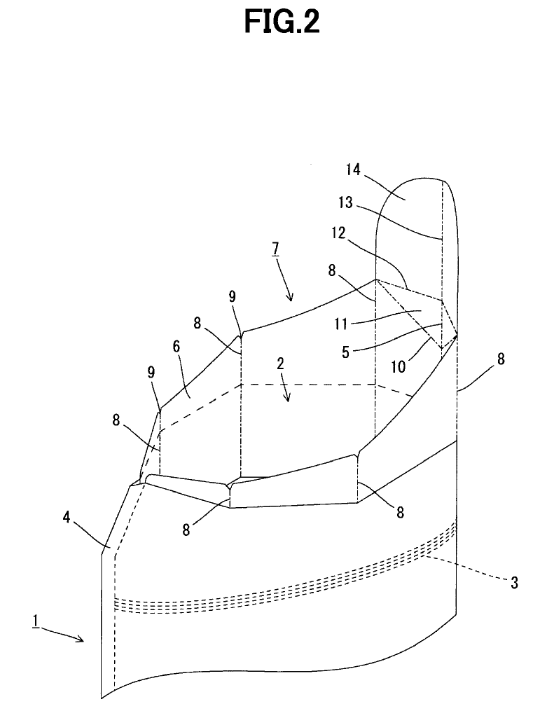

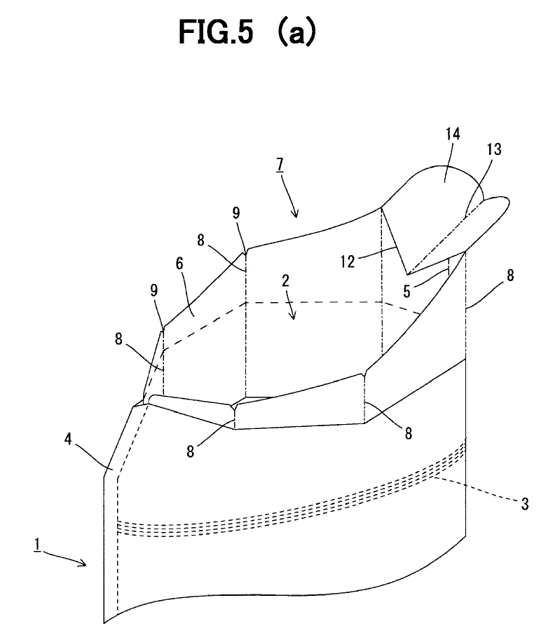

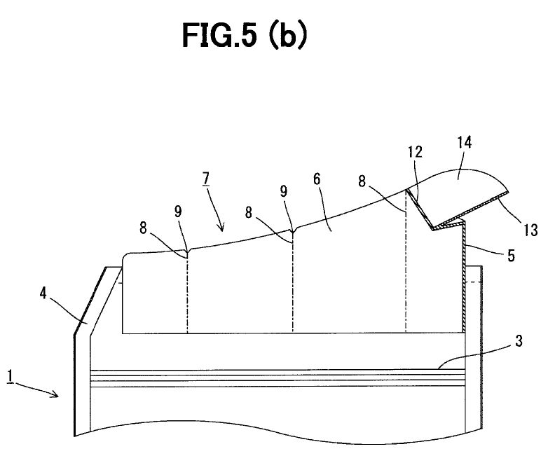

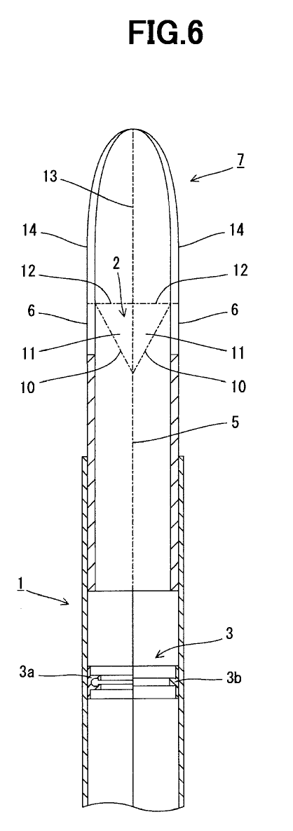

In the condition of the bag as shown in FIG. 2, i.e. the wings 6 and tongues 14, 14 are separated from each other and the opening 2 of the bag defines the substantially cylindrical configuration, pieces 11 defined between the lines 5, 10, and 12 are folded outwardly around the folding lines 10, and the tongues 14, 14 are folded inwardly as shown in FIG. 5. In this condition, only the tip portions of the tongues extend inwardly of the opening, and the other portions of the tongues extend outwardly from the opening. These outwardly extending portions can be used for grasping and supporting the bag when the bag is intended to be used for infants.

In other words, the tongues 14, 14 and the pieces 11 can be used conveniently so that the degree of freedom can be enhanced.

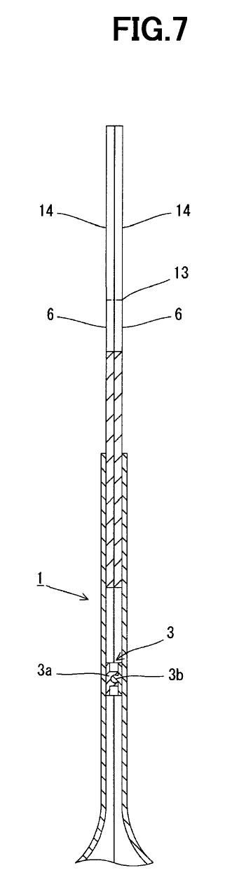

After used the bag, the tongues 14, 14 and the pieces 11 are returned as before as shown in FIG. 2, and the male part 3a and the female part 3b of the sealing fastener 3 are engaged again to seal the bag 1 (see FIG. 7).

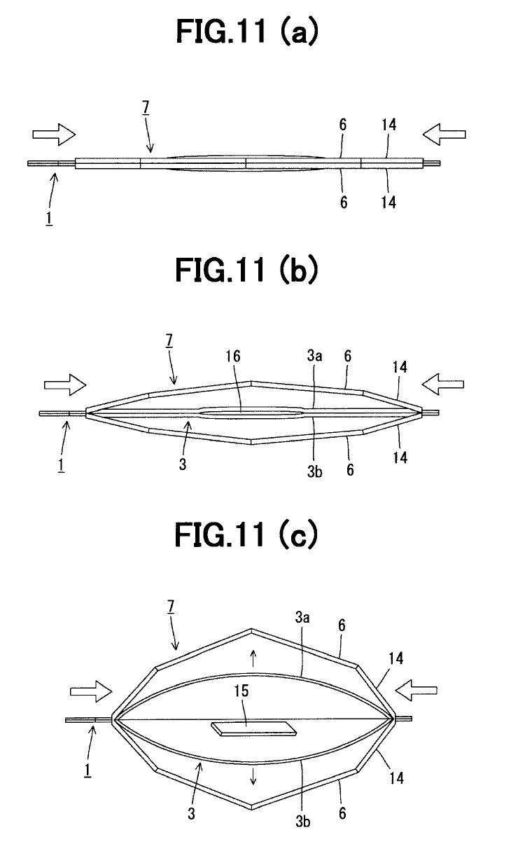

Upon packaging the urine bag including the fastener 3 of the dimensional ratio (W:H) of about 2:1, and the sealing fastener 3 is not closed completely and held partially opened by remaining the unengaged portion 16 between the male part and the female part of the fastener, the bag can be opened easily by the one-touch operation. When it is intended to use the bag, the end of the mouse portion 7 defined by the central folding line 5 and the end opposites thereof are urged toward each other as shown in FIG. 11(a) (see arrows illustrated therein) to deform the mouse portion 7 to separate the opposite wings 6, 6 as shown in FIG. 11(b). Then the male part 3a and a female part 3b of the fastener 3 are separated from the unengaged portion 16 and sheet materials attached thereto are also separated as shown in FIG. 11(c).

Although the unengaged portion 16 can be positioned in any suitable position on the linear fastener, it may be preferable to position it in almost central part of the fastener.

The urine delivered into the bag 1 is adapted to be absorbed and held by water absorbing means 15 to prevent the urine from leaking off while the bag 1 is disposed of after used.

The water absorbing means may include a water absorbent high polymer and a water permeable wrapper material such as of Japanese paper for enclosing the polymer powder. Upon delivered the urine into the bag, it will contact the water absorbing means, and the high polymer contained therein expands, and thus the wrapper will be ruptured and torn to expose the high polymer directly to the urine to enhance the water absorbing effect to be derived. However, in an unused condition, no effects are to be provided on the water absorbing means so that the scattering of the polymer powder contained therein can not be effected.

In the structure of the bag of the embodiment as described above, the mouse portion 7 is formed of cardboard material or paper. The mouse portion 7 can be made of another material such as a cardboard applied on its surface with any suitable coating material. When used is the cardboard material applied on its surface with the material substantially the same as that of the bag portion of the bag 1, it can be connected easily to the bag portion by means of heatsealing. Further, it can achieve water proof Characteristics.

The mouse portion 7 can be made of any blank material of sheet shape such as a sheet of synthetic resin or synthetic paper provided that the material can be folded along the desired folding lines impressed thereon and can hold the desired configuration.

The number of the auxiliary folding lines 8 may be one, two, or four depending on the width of the opening 2 upon used. The purpose of the notches 9 formed at the upper end of each auxiliary folding line 8 is to make it easy to fold the mouse portion 7 along the lines 8. However, depending on the material to be employed in the mouse portion 7, no notches can be made thereon.

Additional notch 9 can be made at the upper end of the second folding line 13 for making it easy to open the tongues 14, 14.

The mouse portion 7 can be fixed to the outer surfaces rather than the inner surfaces of the bag. The mouse portion 7 can surround a portion of the upper part of the bag portion rather than the whole of the opening of the bag portion. When only a portion of the upper part of the bag portion is surrounded by the mouse portion, the mouse portion may have shorter wings. Of course, plurality of shorter mouse portions can be provided around the opening of the bag.

The bag of the present invention can also be used for storing vomit and excreta.

It is to be appreciated that the invention has been described hereinabove with reference to certain examples or embodiments of the invention but that various additions, deletions, alterations and modifications may be made to those examples and embodiments without departing from the intended spirit and scope of the invention. For example, any element or attribute of one embodiment or example may be incorporated into or used with another embodiment or example, unless to do so would render the embodiment or example unpatentable or unsuited for its intended use. All reasonable additions, deletions, modifications and alterations are to be considered equivalents of the described examples and embodiments and are to be included within the scope of the following claims.

What is claimed is:

1. A urine bag comprising:
a bag portion formed of any material inhibiting the permeation of water therethrough, and
a mouse portion to be secured at an upper opening of the bag portion,
the mouse portion is formed of a sheet material surrounding at least a portion of the upper opening and having any suitable flexibility and shape preserving capability,
the mouse portion includes a pair of left and right wings, the wings can be displaced around the central folding line to open or close the upper opening,
the mouse portion further includes a pair of left and right tongues, the tongues can be displaced together with the wings around the second central folding line which is the extension of the central folding line,
the mouse portion is also provided with a pair of symmetrical folding lines extending diagonally from the intersection of the contour of tongues and the upper edges of the wings to the central line.

2. The urine bag as claimed in claim 1 wherein:
the central folding line, the second folding line, and the folding lines are all formed as mountain lines.

3. The urine bag as claimed in claim 1 wherein:
each wing provided with auxiliary folding line or lines extending parallel to the central folding line.

4. The urine bag as claimed in claim 3 wherein:
the auxiliary folding line or lines are also formed as mountain lines.

5. The urine bag as claimed in claim 3 wherein:
the folding line or lines are provided at its or their upper end or ends with a notch or notches.

6. The urine bag as claimed in claim 1 wherein:
the opposite inner faces of the upper opening of the bag portion are provided along their full width with a linear sealing fastener including a male part and a female part engage-able and disengageable with each other.

7. The urine bag as claimed in claim 6 wherein:
the ratio defined between the dimension between fully separated wings and the distance between the lower edge of the mouse portion and the fastener is to be determined as about 2:1.

8. The urine bag as claimed in claim 1 wherein:
a water absorbing means for absorbing urine is contained within the bag.

9. The urine bag as claimed in claim 1 wherein:
the mouse portion is formed of cardboard material.

10. A urine bag comprising:
a bag portion formed of any material inhibiting the permeation of water therethrough, and
a mouse portion to be secured at an upper opening of the bag portion,
the mouse portion is formed of a sheet material surrounding at least a portion of the upper opening and having any suitable flexibility and shape preserving capability,
the mouse portion includes a pair of left and right wings, the wings can be displaced around the central folding line to open or close the upper opening,
the opposite inner faces of the upper opening of the bag portion are provided along their full width with a linear sealing fastener including a male part and a female part engageable and disengageable with each other.
the ratio defined between the dimension between fully separated wings and the distance between the lower edge of the mouse portion and the fastener is to be determined as about 2:1.

11. The urine bag as claimed in claim 10 wherein:
the mouse portion further includes a pair of left and right tongues, the tongues can be displaced together with the wings around the second central folding line which is the extension of the central folding line,
the mouse portion is also provided with a pair of symmetrical folding lines extending diagonally from the intersection of the contour of tongues and the upper edge of the wing to the central line.

12. The urine bag as claimed in claim 11 wherein:
the central folding line, the second folding line, and the folding lines are all formed as mountain lines.

13. The urine bag as claimed in claim 10 wherein:
each wing provided with auxiliary folding line or lines extending parallel to the central folding line.

14. The urine bag as claimed in claim 13 wherein:
the auxiliary folding line or lines are also formed as mountain lines.

15. The urine bag as claimed in claim 13 wherein:
the folding line or lines are provided at its or their upper end or ends with a notch or notches.

16. The urine bag as claimed in claim 10 wherein:
a water absorbing means for absorbing urine is contained within the bag.

17. The urine bag as claimed in claim 10 wherein:
the mouse portion is formed of cardboard material.
